(19) United States
(12) Patent Application Publication
(10) Pub. No.: US 2022/0143070 A1
(43) Pub. Date: May 12, 2022

(54) COMPOSITION FOR USE IN THE PREVENTION AND/OR TREATMENT OF DYSBIOSIS

(71) Applicants: MATIVA TECH, Satigny (CH); INSTITUT NATIONAL DE RECHERCHE POUR L'AGRICULTURE, L'ALIMENTATION ET L'ENVIRONNEMENT, Paris (FR)

(72) Inventors: Hélène DUMINY, CAGNES SUR MER (FR); Alain VERNEAU, POITIERS (FR); Luc HEITZ, LYON (FR); Joël DORÉ, VITRY-SUR-SEINE (FR)

(73) Assignees: MATIVA TECH, Satigny (FR); INSTITUT NATIONAL DE RECHERCHE POUR L'AGRICULTURE, L'ALIMENTATION ET L'ENVIRONNEMENT, Paris (FR)

(21) Appl. No.: 17/435,520

(22) PCT Filed: Mar. 5, 2020
(86) PCT No.: PCT/EP2020/055884

§ 371 Date: Sep. 1, 2021

(30) Foreign Application Priority Data
Mar. 5, 2019 (FR) FR1902224

(51) Int. Cl. A61K 31/717; A61K 31/198; A61K 35/747; A61K 31/20; A61K 45/06; A23L 33/12; A23L 33/175; A23L 33/24; A23L 33/135

(52) U.S. Cl. CPC A61K 31/717; A61K 31/198; A61K 35/747; A61K 31/20; A23L 33/135; A23L 33/12; A23L 33/175; A23L 33/24; A61K 45/06

(57) ABSTRACT

A nutraceutical composition including one or more sources of fiber, glutamine, one or more probiotics, one or more sources of omega-3, and one or more antioxidants, for use in the prevention and/or treatment of dysbiosis of the intestinal microbiota. Also, a method for preventing and/or treating dysbiosis including a step of administering the nutraceutical composition to a patient in need thereof. Further, a three-part kit for use in the prevention and/or treatment of dysbiosis of the intestinal microbiota.

### FIELD OF INVENTION

The present invention relates to a nutraceutical or pharmaceutical composition comprising one or more sources of fiber, glutamine, one or more probiotics, one or more sources of omega-3, and one or more antioxidants, for use in the prevention and/or treatment of dysbiosis of the intestinal microbiota.

The present invention also relates to a method of preventing and/or treating dysbiosis comprising a step of administering the composition according to the invention to a patient in need thereof.

The present invention also relates to a three-part kit for use in the prevention and/or treatment of dysbiosis of the intestinal microbiota.

### BACKGROUND OF INVENTION

The intestinal microbiota is made up of billions of bacteria that live in symbiosis with our body. It is estimated that the microbiota is made up of about 200 species of bacteria. This microbiota has several functions:
It contributes in particular to the degradation of digestible compounds, dietary fibers and polyphenols. It contributes thereby to an essential supply of vitamins from B and K groups, participates in the elimination of carcinogenic compounds and provides the body with short-chain fatty acids (acetate, propionate and butyrate), products resulting from fermentation, which are sources of energy for body tissues
It also exerts a competitive exclusion function over microorganisms from the environment, of which it prohibits the implantation and proliferation by occupying metabolic and ecological niches and by the direct action of antimicrobial molecules.
It also interacts with the intestinal epithelium and beyond with immunity and all organs, thereby modulating trophic development of the intestinal wall and mucus production, immune system tone, liver metabolism, and the function of distal organs such as fatty tissue, muscles and the brain.

For a given individual, the microbiota normally changes little over time, apart from dietary changes or treatments. The homeostasis of the symbiosis is a guarantee of maintaining health and well-being.

Many chronic non-contagious diseases whose incidence has steadily increased since the end of World War II may be associated with impaired microbiota. These include Crohn's disease, obesity, type 1 and 2 diabetes, asthma and multiple sclerosis.

Likewise, psychic or psychological stress can also influence the quality of the microbiota.

Alterations in the microbiota (dysbiosis) can be of different types:
decrease in bacterial diversity,
modification of intestinal permeability,
increase of oxidative stress,
inflammation.

Current therapeutic approaches aim to treat alterations of the microbiota by acting punctually and on only one of the above-indicated alterations.

However, the disadvantage of these approaches is that they do not allow an optimal return to the symbiosis of the microbiota since it is disturbed by several factors that act, in a detrimental way, in parallel.

Therefore, there is a need to develop innovative approaches which, by activating several levers in parallel, would ensure the preservation and/or restoration of the symbiosis of the microbiota.

The Applicant has developed an original approach consisting in combining in the same composition at least one source of dietary fiber, glutamine, at least one probiotic, at least one source of omega-3, and at least one antioxidant. This global approach makes it possible to act on the various levers of dysbiosis and therefore gradually restore the symbiosis.

Moreover, the original approach developed by the Applicant should make it possible to limit the aggravation of chronic diseases, and therefore should allow for the reduction or non-increase in the intake of pharmaceutical products and the associated side-effects.

The composition according to the invention in particular prevent and/or treat dysbiosis of the intestinal microbiota, and thus preserve and/or restore the symbiosis of the intestinal microbiota.

### SUMMARY

The invention thus relates to a nutraceutical or pharmaceutical composition comprising
a. one or more sources of dietary fibers,
b. glutamine,
c. one or more probiotics,
d. one or more sources of omega-3, and
e. one or more antioxidants,
for use in the prevention and/or treatment of dysbiosis of the intestinal microbiota.

In one embodiment, the source(s) of dietary fibers of the composition according to the invention are selected from sources of vegetable fibers. In one embodiment, the source(s) of dietary fibers of the composition according to the invention are selected from sources of celluloses, hemicellulose, pectins, starch, beta-glucans, and a mixture of these compounds.

In one embodiment, the probiotic(s) of the composition according to the invention are selected from lactic acid bacteria. In one embodiment, the probiotic(s) of the composition according to the invention are selected from the family of Lactobacillaceae, Bifidobacteriaceae, and a mixture thereof. In one embodiment, the probiotic(s) of the composition according to the invention are selected from Lactobacillus rhamnosus, Lactobacillus plantarum, Bifidobacterium breve, and a mixture thereof.

In one embodiment, the source(s) of omega-3 of the composition according to the invention are selected from docosahexaenoic acid, eicosatetraenoic acid, hexadecatrienoic acid, α-linolenic acid, stearidonic acid, eicosatrienoic acid, eicosapentaenoic acid, heneicosapentaenoic acid, docosapentaenoic acid, tetracosapentaenoic acid, tetracosahexaenoic acid, and mixtures of these compounds. In one embodiment, the source(s) of omega-3 of the composition according to the invention are selected from docosahexaenoic acid, eicosatetraenoic acid, α-linolenic acid, and mixtures of these compounds.

In one embodiment, the antioxidant(s) of the composition according to the invention are selected from curcuminoids, quercetin, rutin, catechins, boswellic acid, resveratrol, berberine, silybin and a mixture of these compounds. In one embodiment, the antioxidant(s) of the composition according to the invention are selected from curcumin, quercetin, resveratrol, berberine, silybin, catechins, boswellic acid and a mixture of these compounds.

In one embodiment, the composition according to the invention is in three parts, a first part comprising the source(s) of dietary fibers and glutamine, a second part comprising the probiotic(s), a third comprising the source(s) of omega-3, the antioxidant(s) being included in the first part of the composition and/or in the third part of the composition. In one embodiment, the antioxidant(s) are included in the first part and in the third part of the composition

In one embodiment, the first part of the composition according to the invention has a total content of sources of fibers ranging from 30 to 70% by weight, relative to the total weight of the first part of the composition. In one embodiment, the first part of the composition according to the invention has a total content of sources of fibers ranging from 40 to 60% by weight, relative to the total weight of the first part of the composition.

In one embodiment, the first part of the composition according to the invention has a total content of glutamine ranging from 30 to 70% by weight, relative to the total weight of the first part of the composition. In one embodiment, the first part of the composition according to the invention has a total content of glutamine ranging from 40 to 60% by weight, relative to the total weight of the first part of the composition.

In one embodiment, the third part of the composition according to the invention has a total content of sources of omega-3 ranging from 10 to 50% by weight, relative to the total weight of the third part of the composition. In one embodiment, the third part of the composition according to the invention has a total content of sources of omega-3 ranging from 20 to 40% by weight, relative to the total weight of the third part of the composition.

In one embodiment, the composition according to the invention is for use in the prevention and/or treatment of dysbiosis of the intestinal microbiota associated with Irritable Bowel Syndrome (IBS), type I diabetes, type II diabetes, obesity, non-alcoholic fatty liver disease (NAFLD) and Crohn's disease. In one embodiment, the composition according to the invention is for use in the prevention and/or treatment of dysbiosis of the intestinal microbiota associated with Irritable Bowel Syndrome (IBS), type I diabetes, type II diabetes, obesity and non-alcoholic fatty liver disease (NAFLD). In one embodiment, the composition according to the invention is for use in the prevention and/or treatment of dysbiosis of the intestinal microbiota associated with Irritable Bowel Syndrome (IBS).

### Definitions

In the present invention, the terms below are defined as follows:
“Antioxidant capacity” refers to a biological parameter which reports on the antioxidant protection system and constitutes a method for detecting oxidative stress. The antioxidant capacity is lower as the production of free radicals is higher.
“About” when it precedes a number, means plus or minus 10% of the value of said number.
“Dysbiosis of the intestinal microbiota” refers to an imbalance or poor microbial adaptation of the intestinal microbiota. It can in particular be manifested by one or more of the following events: decrease in bacterial diversity, change in intestinal permeability, increase in oxidative stress and inflammation. In one embodiment, dysbiosis is manifested by at least two of the following events: decrease in bacterial diversity, change in intestinal permeability, increase in oxidative stress, and low-grade inflammation (as distinguished from acute inflammation). In one embodiment, dysbiosis is manifested by at least three of the following events: decrease in bacterial diversity, change in intestinal permeability, increase in oxidative stress, and low-grade inflammation. In one embodiment, dysbiosis is manifested by all of the following events: decreased bacterial diversity, change in intestinal permeability, increase in oxidative stress, and low-grade inflammation.
“Nutraceutical composition” or “Functional food” or “Foods with nutritional purposes” or “Food supplements” are interchangeable terms and refers to any substance containing nutrients, whether intended for human or animal consumption, whether composed of a single ingredient or of a mixture of ingredients, whether liquid, semi-liquid or solid, whether it mainly comprises carbohydrates, fats, proteins or any mixture thereof, whether it is edible in itself or requiring treatment such as cooking, mixing, cooling, mechanical treatment, etc.
“Pharmaceutical composition” refers to a composition comprising an active agent in combination with a pharmaceutically acceptable vehicle or excipient. A pharmaceutical composition is for therapeutic use and relates to health. In particular, a pharmaceutical composition may be indicated for treating or preventing a disease. According to the invention, the term “treatment of a disease” refers to the reduction or alleviation of at least one adverse effect or symptom of a disease, disorder or condition associated with a deficiency of the function of an organ, tissue or cell. The term “preventing disease” or “inhibiting the development of disease” refers to the prevention or avoidance of the onset of a symptom.
“Adverse effect” refers to any harmful reaction occurring in a patient, a living donor or a recipient, related or likely to be related to a medicinal product.
“Serious adverse effect” refers to any adverse effect that is lethal, or likely to endanger life, or causes significant or lasting disability or incapacity, or causes or prolongs hospitalization, or manifests itself in a congenital anomaly or malformation.
“Adverse event” refers to any harmful event occurring in a person who lends itself to biomedical research, whether or not this event is related to the research or to the product on which this research relates.
“Serious adverse event” refers to any adverse event which: results in death; endangers the life of the person who submits to the research; requires hospitalization or prolongation of hospitalization; causes significant or lasting disability or handicap; results in a congenital anomaly or malformation; or any event considered medically serious, and with regard to the drug, regardless of the administered dose.
“Dietary fibers” refers to the parts of an element of plant origin made up of complex mixtures of carbohydrates from the cell wall or cytoplasm of vegetable cells, and which cannot be completely broken down by human digestive enzymes. In one embodiment, the dietary fibers, soluble or insoluble, are chosen from pectins, celluloses, hemicelluloses, starches, in particular resistant starches, beta-glucans and a mixture of these compounds. “Sources of dietary fibers” refers to foods or nutrients comprising dietary fiber. These include vegetable foods such as fruits, vegetables, legumes and grains. Regarding fruits as sources of dietary fiber, mention may in particular be made of almonds, currants, prunes, walnuts and bananas. Regarding vegetables as sources of dietary fiber, mention may in particular be made of artichokes, carrots, potatoes, green cabbage and cauliflower. Regarding legumes as a source of dietary fiber, mention may in particular be made of white beans, split peas, chickpeas, lentils and peas. Regarding cereals as sources of dietary fiber, mention may in particular be made of wheat bran, oat bran, oatmeal and rice. In one embodiment, sources of dietary fibers will denote foods or nutrients comprising at least 3 g of fibers per 100 g of food or nutrient, preferably comprising at least 6 g of fibers per 100 g of food. or nutrient.
“Inflammation” refers to a response of living tissues to an attack, of which the etiology may, for example, be infectious, physical, chemical, vascular, dysimmunitary. It involves cells, vessels, changes in the extracellular matrix and many chemical mediators of inflammation. “Acute inflammation” refers to the immediate response to an aggressive agent, of short duration (a few days or weeks), of often sudden onset and characterized by intense vasculo-exudative phenomena.

“Low-grade inflammation” or “chronic inflammation” refers to an inflammation that is opposed to acute inflammation in that its onset is slower, in that the biological and clinical signs are less intense and in that it evolves persisting or worsening for several months or years.
“Probiotic” refers to living microorganisms (bacteria or yeasts) added exogenously to an organism, for example via food, which have a beneficial effect on the organism which ingests them.
“Omega-3” refers to omega-3 fatty acids that are polyunsaturated fatty acids including at least one carbon-carbon double bond between the 3^{rd} and the 4^{th} carbon of the carbon chain of the acid, starting with the last carbon of the carbon chain (omega carbon).
“Antioxidant” refers to a molecule that slows down or prevents the oxidation process, i.e., the oxidation of other chemical substances in particular due to the presence of free radicals by neutralizing them. For the purposes of the present invention, the term “antioxidant” is aimed in particular at a molecule which slows down or prevents the process of oxidation of human cells.
“Glutamine” refers to the amino acid of formula:

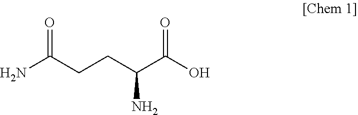

“FIB4 score” or “Fibrosis-4 score” refers to a score used to assess fibrosis, including the following parameters: patient age, serum ASAT and ALT concentrations, venous blood platelet concentration.
“NAFLD Score” or “Non-Alcoholic Fatty Liver Disease Fibrosis Score” or “NFS score” refers to a score used to assess fibrosis, including the following parameters: patient age, hyperglycemia/diabetes, body mass index (BMI), serum concentrations of ASAT, ALT and albumin, venous blood platelet concentration.
“SAF score” (Steatosis Activity Fibrosis) refers to a composite histological score taking into account steatosis, histological activity and hepatic fibrosis, as defined in Bedossa, P. et al., “Histopathological algorithm and scoring system for evaluation of liver lesions in morbidly obese patients”, Hepatology, November 2012, 56 (5): 1751-9.
“Oxidative stress” designates an attack on cells by free radicals, also called “reactive oxygen species” (ROS), by nitrogen monoxide NO, by hydrogen peroxide H₂O₂ or by the hydroxyl radical HO^{⋅}. These compounds attacking cells lead to peroxidation of proteins and lipids, or even to an alteration of nucleic acids (DNA breaks, etc.). For the purposes of the present invention, the cells attacked during oxidative stress are in particular human cells.
“Subject” designates an animal, including a human being. For the purposes of the present invention, a subject may be a patient, namely a person receiving medical care, undergoing or having undergone medical treatment, or being monitored for the development of a disease. Said disease may for example be non-alcoholic fatty liver disease (NAFLD), Irritable Bowel Syndrome (IBS), type I diabetes, type II diabetes, obesity, Crohn's disease, preferably an non-alcoholic fatty liver disease (NAFLD).
“Symbiosis of the intestinal microbiota” means, in opposition to dysbiosis, a balance between the intestinal microbiota and the body.

### DETAILED DESCRIPTION

The invention relates to a nutraceutical or pharmaceutical composition comprising:
a. one or more sources of dietary fibers,
b. glutamine,
c. one or more probiotics,
d. one or more sources of omega-3, and
e. one or more antioxidants,
for use in the prevention and/or treatment of dysbiosis of the intestinal microbiota.

The Applicant has found out that the composition according to the invention makes it possible to prevent and/or treat dysbiosis of the intestinal microbiota.

By acting on the four axes identified in parallel:
increase in bacterial diversity,
decrease in intestinal permeability,
reduction of oxidative stress, and
decrease in low-grade inflammation,
the composition according to the invention makes it possible to restore the symbiosis of the intestinal microbiota and the body.

Sources of Dietary Fibers

The composition according to the invention comprises one or more sources of dietary fiber. In one embodiment according to the invention, the dietary fibers serve as the primary substrate of the microbial trophic chain. They constitute thereby an ingredient of choice for opening up ecological niches and enabling a great diversity of microorganisms suitable for hydrolyzing various complex polysaccharide structures to become dominant.

In one embodiment, the sources of dietary fibers are selected from sources of vegetable fibers.

In one embodiment, the sources of dietary fibers are selected from sources of soluble dietary fibers.

In one embodiment, the sources of dietary fibers are selected from sources of insoluble dietary fiber.

In one embodiment, the sources of dietary fibers are selected from sources of celluloses, hemicellulose, starch, pectins, beta-glucans, and a mixture of these compounds.

In one embodiment, the sources of fibers can be selected from fruits, vegetables, legumes, cereals or a mixture of these sources.

In one embodiment, the fruits are selected from almonds, currants, prunes, walnuts, bananas and a mixture of these sources.

In one embodiment, the vegetables are selected from artichokes, carrots, potatoes, green cabbages, cauliflowers, and a mixture of these sources.

In one embodiment, the legumes are selected from white beans, split peas, chickpeas, lentils, peas, and a mixture of these sources.

In one embodiment, the cereals are selected from wheat bran, oat bran, oatmeal, rice, and a mixture of these sources.

In one embodiment, the sources of dietary fibers that can be used according to the invention are directly dietary fibers as defined above.

Thus, in one embodiment, the sources of dietary fibers are chosen from celluloses, hemicelluloses, starches, pectins, beta-glucans, and a mixture of these compounds.

In one embodiment, the sources of dietary fibers are celluloses.

In one embodiment, the sources of dietary fibers are hemicelluloses.

In one embodiment, the sources of dietary fibers are starches. In one embodiment, the sources of dietary fibers are resistant starches.

In one embodiment, the sources of dietary fibers are pectins.

In one embodiment, the sources of dietary fibers are beta-glucans.

In one embodiment, the sources of dietary fibers are a mixture of celluloses, pectins, and beta-glucans.

In one embodiment, the sources of dietary fibers comprise at least 3 g of fibers per 100 g of sources of dietary fibers.

In one embodiment, the sources of dietary fibers comprise at least 6 g of fibers per 100 g of sources of dietary fibers.

In one embodiment, the sources of dietary fibers are directly dietary fibers and comprise 100 g of fibers per 100 g of dietary fibers sources.

Glutamine

The composition according to the invention comprises glutamine. In one embodiment, the term “glutamine” denotes the amino acid of formula:

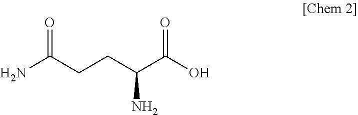

i.e., L or S(+)-glutamine.

In one embodiment, glutamine helps maintain and/or restore the intestinal barrier. Indeed, dysbiosis can result in hyperpermeability of the intestinal wall.

In one embodiment, glutamine is an energy source for intestinal epithelial cells.

Glutamine is commercially available in different pack sizes and in different amounts.

Probiotics

The composition according to the invention comprises one or more probiotics. In one embodiment, the probiotics help reduce inflammation. In one embodiment, the probiotics help restore intestinal permeability. In one embodiment, the probiotics have an action on the composition of the microbiota. In one embodiment, the probiotics increase the diversity of the composition of the microbiota.

In one embodiment, the probiotic(s) are selected from lactic acid bacteria.

In one embodiment, the probiotic(s) are selected from the family of Lactobacillaceae, Bifidobacteriaceae, and a mixture thereof.

In one embodiment, the probiotic(s) of the composition according to the invention are selected from Lactobacillus rhamnosus, preferably Lactobacillus rhamnosus GG, Lactobacillus plantarum, preferably Lactobacillus plantarum LP01, Bifidobacterium breve, preferably Bifidobacterium breve BR03, and a mixture of these probiotics.

In one embodiment, the probiotic(s) of the composition according to the invention are the mixture consisting of Lactobacillus rhamnosus GG, Lactobacillus plantarum LP01 and Bifidobacterium breve BR03. Advantageously, this mixture of probiotics is present in the composition according to the invention in an amount of 5 billion CFU (daily amount), all strains of probiotics included. For example, if each strain is dosed at 100 billion CFU per gram, the composition includes 50 mg of probiotics comprising the three strains of probiotics.

In one embodiment, the probiotic of the composition according to the invention is Lactobacillus rhamnosus, preferably Lactobacillus rhamnosus GG.

In one embodiment, the number of colony-forming unit (or “CFU”) of probiotics is greater than or equal to 10⁶ cells per gram of probiotics. In one embodiment, the number of colony-forming units of probiotics is greater than or equal to 5.10⁶ cells per gram of probiotics. In one embodiment, the number of colony-forming units of probiotics is greater than or equal to 10⁷ cells per gram of probiotics. In one embodiment, the number of colony-forming units of probiotics is greater than or equal to 5.10⁷ cells per gram of probiotics. In one embodiment, the number of colony-forming units of probiotics is greater than or equal to 10⁸ cells per gram of probiotics. In one embodiment, the number of colony-forming units of probiotics is greater than or equal to 5.10⁸ cells per gram of probiotics. In one embodiment, the number of colony-forming units of probiotics is greater than or equal to 10⁹ cells per gram of probiotics.

In one embodiment, the number of colony-forming units of probiotic is between 10⁶ and 10¹⁰ cells per gram of probiotics.

Omega-3 Sources

The composition according to the invention comprises one or more sources of omega-3. In one embodiment, the omega-3 help reduce inflammation. In one embodiment, the omega-3 help restore intestinal permeability. In one embodiment, the omega-3 have an action on the composition of the microbiota. In one embodiment, the omega-3 increase the compositional diversity of the microbiota.

In one embodiment, the source(s) of omega-3 is selected from fish oils such as salmon, halibut, herring, anchovies and sardines.

In one embodiment, the source(s) of omega-3 are selected from vegetable oils such as vegetable oils obtained from flax seeds, chia seeds, and hemp seeds, rapeseed oil and walnut oil.

In one embodiment, the source(s) of omega-3 are selected from algae.

In one embodiment, the source(s) of omega-3 are directly omega-3.

In one embodiment, the source (s) of omega-3 are selected from omega-3 polyunsaturated fatty acids comprising 18, 19, 20, 21, 22, 23, 24 or 25 carbon atoms, preferably 21, 22 or 23 carbon atoms.

In one embodiment, the source or sources of omega-3 are selected from docosahexaenoic acid, eicosatetraenoic acid, hexadecatrienoic acid, α-linolenic acid, stearidonic acid, eicosatrienoic acid, eicosapentaenoic acid, heneicosapentaenoic acid, docosapentaenoic acid, tetracosapentaenoic acid, tetracosahexaenoic acid, and mixtures of these compounds.

In one embodiment, the source(s) of omega-3 are selected from docosahexaenoic acid, eicosatetraenoic acid, α-linolenic acid, and mixtures of these compounds.

Antioxidants

The composition according to the invention also comprises one or more antioxidants. In one embodiment, the antioxidant(s) decrease inflammation. In one embodiment, the antioxidant(s) reduce oxidative stress. In one embodiment, the antioxidant(s) reduce inflammation and oxidative stress. In one embodiment, the antioxidant(s) contribute to the restoration of intestinal permeability. In one embodiment, the antioxidant(s) have an action on the composition of the microbiota. In one embodiment, the antioxidant(s) increase the diversity of the composition of the microbiota.

In one embodiment, the antioxidant(s) are selected from curcuminoids such as curcumin, quercetin, rutin, catechins, boswellic acid, resveratrol, berberine, silybin and a mixture of these compounds.

In one embodiment, the antioxidant(s) are selected from curcumin, quercetin, resveratrol, berberine, silybin, catechins, boswellic acid and a mixture of these compounds

In one embodiment, Boswellia is used as a source of boswellic acid.

In one embodiment, green tea is used as a source of catechins.

In one embodiment, milk thistle is used as a source of silybin.

In one embodiment, turmeric is used as a source of curcumin. In one embodiment, Sophora japonica is used as a source of quercetin.

According to one embodiment, the antioxidant(s) is a mixture, administered to the subject in need thereof via a single dosage form or administered by distributing the antioxidants in at least two dosage forms, comprising at least two compounds selected from:
silybin, or dry extract of aerial parts of milk thistle (Silybum marianum) preferably titrated at 80% by weight of silybin,
quercetin,
curcumin, or extract of turmeric rhizome preferably titrated from 80% to 95% by weight in curcumin, and/or
vitamin E.

According to one preferred embodiment, the antioxidant(s) is a mixture, administered to the subject in need thereof via a single dosage form or administered by distributing the antioxidants in at least two dosage forms, consisting of at least two compounds selected from:
silybin, or dry extract of aerial parts of milk thistle (Silybum marianum) preferably titrated at 80% by weight of silybin,
quercetin,
curcumin, or extract of turmeric rhizome preferably titrated from 80% to 95% by weight in curcumin, and/or
vitamin E.

In prior art, certain antioxidants have been tested individually and at high doses in the treatment of NAFLD; the interest of the specific mixture consisting of at least two compounds among silybin, or dry extract of aerial parts of milk thistle (Silybum marianum) preferably titrated at 80% by weight of silybin; quercetin; curcumin, or turmeric rhizome extract preferably titrated from 80% to 95% by weight in curcumin; and/or vitamin E, is that it allows synergistic action of said antioxidants in improving NAFLD.

Indeed, the antioxidants are used individually at doses lower than the individual doses used in prior art, and the combination of this particular mixture of antioxidants within a composition according to the present invention leads to a greater effect in the treatment. of NAFLD than the effect of each individual antioxidant tested at high dose in prior art.

In one embodiment, the antioxidant(s) is a mixture administered in one or more pharmaceutical forms, for example for use within the composition according to the invention in the prevention and/or treatment of non-alcoholic fatty liver disease, and is composed of a total daily dose of:
less than 1000 mg, preferably less than 500 mg, more preferably less than 300 mg of dry extract of aerial parts of milk thistle (Silybum marianum) titrated to 80% by weight of silybin,
less than 1000 mg, preferably less than 500 mg, more preferably less than 300 mg of quercetin,
less than 1.75 g, preferably less than 1.5 g, more preferably less than 1.35 g of turmeric rhizome extract titrated from 80% to 95% by weight as curcumin, and
less than 600 IU, preferably less than 100 IU, more preferably less than 20 IU of vitamin E.

In one embodiment, the antioxidant(s) is a mixture administered in one or more pharmaceutical forms, for example for use within the composition according to the invention in the prevention and/or treatment of non-alcoholic fatty liver disease, and is composed of a total daily dose of:
200 mg of dry extract of aerial parts of milk thistle (Silybum marianum) titrated at 80% by weight of silybin,
100 mg of quercetin,
1 g of turmeric rhizome extract titrated to at least 80% by weight in curcumin, preferably titrated from 80% to 95% by weight in curcumin, and
12 mg (181 U) of vitamin E.

Alternatively, the antioxidant(s) is a mixture administered in one or more pharmaceutical forms, for example for use within the composition according to the invention in the prevention and/or treatment of type II diabetes, and is composed of a total daily dose of:
less than 5000 mg, preferably less than 1000 mg, more preferably less than 500 mg, even more preferably about 200 mg of berberine,
less than 1000 mg, preferably less than 500 mg, more preferably less than 300 mg, even more preferably about 100 mg of quercetin,
less than 1.75 g, preferably less than 1.5 g, more preferably less than 1.35 g, even more preferably about 1 g of turmeric rhizome extract titrated to at least 80%, preferably 80% at 95% by weight in curcumin,
less than 600 IU, preferably less than 100 IU, more preferably less than 20 IU, even more preferably about 12 mg (18 IU) of vitamin E.

Alternatively, the antioxidant(s) is a mixture administered in one or more pharmaceutical forms, for use within the composition according to the invention in the prevention and/or treatment of type II diabetes, and is composed of a daily dose total of:
less than 1000 mg, preferably less than 500 mg, more preferably less than 300 mg, even more preferably about 200 mg of dry extract of aerial parts of milk thistle (Silybum marianum) titrated to 80% by weight of silybin,
less than 1000 mg, preferably less than 500 mg, more preferably less than 300 mg, even more preferably about 100 mg of quercetin,
less than 1.75 g, preferably less than 1.5 g, more preferably less than 1.35 g, even more preferably about 1 g of turmeric rhizome extract titrated from 80% to 95% by weight of curcumin, and
less than 600 IU, preferably less than 100 IU, more preferably less than 20 IU, even more preferably about 18 IU of vitamin E.

Formulation

In one embodiment, the composition according to the invention comprises or consists of:
glutamine;
silybin or a dry extract of aerial parts of milk thistle (Silybum marianum) preferably titrated at 80% by weight of silybin;
beta-glucans, for example oat beta-glucans;
pectins;
cellulose;
a mixture of three probiotics: Lactobacillus rhamnosus GG, Bifidobacterium breve BR03 (DSM 16604), Lactobacillus plantarum LP01 (LMG P-21021);
fish oil, preferably titrated to at least 60% by weight of omega-3, for example said omega-3 can comprise docosahexaenoic acid (DHA) and eicosapentaenoic acid (EPA);
turmeric or an extract of turmeric rhizome titrated from 80% to 95% by weight in curcumin; and
quercetin.

According to one embodiment, the composition according to the invention also comprises vitamin E, preferably natural vitamin E and/or acacia fibers.

In one embodiment, the composition according to the invention comprises or consists of:
glutamine;
vitamin E, preferably natural vitamin E;
silybin or a dry extract of aerial parts of milk thistle (Silybum marianum) preferably titrated at 80% by weight of silybin;
beta-glucans, for example oat beta-glucans;
pectins;
cellulose;
a mixture of three probiotics: Lactobacillus rhamnosus GG, Bifidobacterium breve BR03 (DSM 16604), Lactobacillus plantarum LP01 (LMG P-21021);
acacia fibers;
fish oil, preferably titrated to at least 60% by weight of omega-3, for example said omega-3 can comprise docosahexaenoic acid (DHA) and eicosapentaenoic acid (EPA);
turmeric or an extract of turmeric rhizome titrated from 80% to 95% by weight in curcumin; and
quercetin.

In one embodiment, the composition according to the invention comprises or consists of:
less than 30 g, preferably less than 15 g, more preferably less than 8 g, even more preferably about 5 g of glutamine;
less than 1000 mg, preferably less than 500 mg, more preferably less than 300 mg, even more preferably about 200 mg of dry extract of aerial parts of milk thistle (Silybum marianum) titrated at 80% by weight of silybin;
less than 10 g, preferably less than 10 g, more preferably less than 2.5 g, even more preferably about 1.666 g of oat beta-glucans;
less than 10 g, preferably less than 5 g, more preferably less than 2.5 g, even more preferably about 1.666 g of pectins;
less than 10 g, preferably less than 5 g, more preferably less than 2.5 g, even more preferably about 1.666 g of cellulose;
less than 100 g, preferably less than 75 g, more preferably less than 55 g, even more preferably about 50 mg (i.e., about 5 billion CFU) of a mixture of three probiotics: Lactobacillus rhamnosus GG, Bifidobacterium breve BR03 (DSM 16604), Lactobacillus plantarum LP01 (LMG P-21021);
less than 2000 mg, preferably less than 1500 mg, more preferably less than 1250 mg, even more preferably about 1000 mg of fish oil, preferably titrated to at least 60% by weight of omega-3, for example said omega-3s may comprise docosahexaenoic acid (DHA) and eicosapentaenoic acid (EPA);
less than 1.75 g, preferably less than 1.5 g, more preferably less than 1.35 g, even more preferably about 1 g of extract of turmeric rhizome (Curcuma longa) titrated at 80% by weight of curcumin; and
less than 1000 mg, preferably less than 500 mg, more preferably less than 300 mg, even more preferably about 100 mg of quercetin.

According to one embodiment, the composition according to the invention also comprises:
less than 600 IU, preferably less than 100 IU, more preferably less than 20 IU, even more preferably about 18 IU (12 mg) of vitamin E, preferably natural vitamin E; and/or
less than 500 mg, preferably less than 400 mg, more preferably less than 300 mg, even more preferably about 200 mg of acacia fibers.

Advantageously, the fish oil of the composition according to the invention comprises 60% by weight of omega-3, in particular about 30% by weight of docosahexaenoic acid (DHA) relative to the weight of the fish oil, about 20% by weight of eicosapentaenoic acid (EPA) based on the weight of fish oil and about 10% by weight of omega-3 other than DHA and EPA based on the weight of fish oil.

The composition according to the invention can in particular be administered once a day to a subject in need thereof.

The various components of the composition according to the invention can be packaged in at least one part. Preferably, each part corresponds to a dosage form which can be administered in one or more dosage units, in particular for once daily administration of each part to a subject in need thereof. For example, the composition according to the invention can be packaged in three parts, one part being packaged in a sachet, one part being packaged in a capsule and one part being packaged in two soft capsules.

In one embodiment, the composition according to the invention is in three parts.

In one embodiment, the composition according to the invention is in three parts:
a first part comprising one or more sources of dietary fibers, preferably as defined above, glutamine, preferably as defined above,
a second part comprising one or more probiotics, preferably as defined above,
a third part comprising one or more sources of omega-3, preferably as defined above,
one or more antioxidants, preferably as defined above, the antioxidant(s) being included in the first part of the composition and/or in the second part of the composition.

In one embodiment, the first and the third part of the composition according to the invention comprise one or more antioxidants.

In one embodiment, only the first part of the composition according to the invention comprise one or more antioxidants.

In one embodiment, only the third part of the composition according to the invention comprise one or more antioxidants.

In one embodiment, the second part of the composition according to the invention does not comprise antioxidants.

In one embodiment, the first part of the composition according to the invention has a total content of sources of fibers ranging from 30 to 70% by weight, relative to the total weight of the first part of the composition according to the invention.

In one embodiment, the first part of the composition according to the invention has a total content of sources of fibers ranging from 40 to 60% by weight, relative to the total weight of the first part of the composition according to the invention.

In one embodiment, the first part of the composition according to the invention has a total content of glutamine ranging from 30 to 70% by weight, relative to the total weight of the first part of the composition according to the invention.

In one embodiment, the first part of the composition according to the invention has a total content of glutamine ranging from 40 to 60% by weight, relative to the total weight of the first part of the composition according to the invention.

In one embodiment, the first part of the composition according to the invention has a total content of antioxidants ranging from 0 to 40% by weight, relative to the total weight of the first part of the composition according to the invention.

In one embodiment, the first part of the composition according to the invention has a total content of antioxidants ranging from 0 to 20% by weight, relative to the total weight of the first part of the composition according to the invention.

In one embodiment, the first part of the composition according to the invention has a total content of antioxidants ranging from 0 to 10% by weight, relative to the total weight of the first part of the composition according to the invention.

In one embodiment, the first part of the composition according to the invention has a total content of antioxidants ranging from 1 to 5% by weight, relative to the total weight of the first part of the composition according to the invention.

In one embodiment, the second part of the composition according to the invention has a total content of probiotics ranging from 80 to 100% by weight, relative to the total weight of the second part of the composition according to the invention.

In one embodiment, the second part of the composition according to the invention has a total content of probiotics ranging from 90 to 100% by weight, relative to the total weight of the second part of the composition according to the invention.

In one embodiment, the second part of the composition according to the invention has a total content of probiotics of 100% by weight, relative to the total weight of the second part of the composition according to the invention.

In one embodiment, the third part of the composition according to the invention has a total content of sources of omega-3 ranging from 10 to 50% by weight, relative to the total weight of the third part of the composition according to the invention.

In one embodiment, the third part of the composition according to the invention has a total content of sources of omega-3 ranging from 20 to 40% by weight, relative to the total weight of the third part of the composition according to the invention.

In one embodiment, the third part of the composition according to the invention has a total content of antioxidants ranging from 50 to 90% by weight, relative to the total weight of the third part of the composition according to the invention.

In one embodiment, the third part of the composition according to the invention has a total content of antioxidants ranging from 60 to 80% by weight, relative to the total weight of the third part of the composition according to the invention.

In one embodiment, the composition according to the invention comprises:
a first part comprising 30 to 70% by weight, preferably from 40 to 60% by weight of sources of fibers, from 30 to 70% by weight, preferably from 40 to 60% by weight of glutamine, and from 0 to 40% by weight, preferably from 0 to 20% by weight, more preferably from 0 to 10% by weight, even more preferably from 1 to 5% by weight of antioxidants, relative to the total weight of the first part,
a second part comprising from 80 to 100% by weight, preferably from 90 to 100% by weight, more preferably 100% by weight of probiotics, relative to the total weight of the second part, and
a third part comprising from 10 to 50% by weight, preferably from 20 to 40% by weight of omega-3, from 50 to 90% by weight, preferably from 60 to 80% by weight of antioxidants relative to the total weight of the third part.

In one embodiment, the first part of the composition represents from 70 to 90% by weight of the composition relative to the total weight of the composition.

In one embodiment, the first part of the composition represents from 80 to 90% by weight of the composition relative to the total weight of the composition.

In one embodiment, the second part of the composition represents from 10 to 19.999% by weight of the composition relative to the total weight of the composition.

In one embodiment, the second part of the composition represents from 12 to 18% by weight of the composition relative to the total weight of the composition.

In one embodiment, the third part of the composition represents from 0.001 to 1% by weight of the composition relative to the total weight of the composition.

In one embodiment, the third part of the composition represents from 0.01 to 1% by weight of the composition relative to the total weight of the composition.

In one embodiment, the third part of the composition represents from 0.1 to 1% by weight of the composition relative to the total weight of the composition.

In one embodiment, the first part/second part weight ratio ranges from 1 to 10. In one embodiment, the first part/second part weight ratio ranges from 3 to 8.

In one embodiment, the first part/second part weight ratio ranges from 5 to 8. In one embodiment, the second part/third part weight ratio varies from 10 to 50. In one embodiment, the second part/third part weight ratio varies from 20 to 40. In one embodiment, the second part/third part weight ratio varies from 30 to 40. In one embodiment, the first part/third part weight ratio varies from 100 to 300.

In one embodiment, the first part/third part weight ratio varies from 150 to 250. In one embodiment, the first part/third part weight ratio varies from 180 to 220.

According to one embodiment, the first part comprises:
glutamine,
vitamin E, preferably natural vitamin E,
silybin or a dry extract of aerial parts of milk thistle (Silybum marianum) preferably titrated at 80% by weight of silybin,
beta-glucans, preferably oat beta-glucans,
pectins, and
cellulose.

Optionally, the first part can also comprise turmeric, or an extract of turmeric rhizome titrated from 80% to 95% by weight in curcumin, and quercetin.

According to a preferred embodiment, the first part comprises:
less than 30 g, preferably less than 15 g, more preferably less than 8 g of glutamine,
less than 600 IU, preferably less than 100 IU, more preferably less than 20 IU of vitamin E, preferably natural vitamin E,
less than 1000 mg, preferably less than 500 mg, more preferably less than 300 mg of dry extract of aerial parts of milk thistle (Silybum marianum) titrated at 80% by weight of silybin,
less than 10 g, preferably less than 10 g, more preferably less than 2.5 g of beta-glucans, preferably oat beta-glucans,
less than 10 g, preferably less than 5 g, more preferably less than 2.5 g of pectins, and
less than 10 g, preferably less than 5 g, more preferably less than 2.5 g of cellulose.

Optionally, the first part can also comprise:
less than 1.75 g, preferably less than 1.5 g, more preferably less than 1.35 g of turmeric rhizome extract (Curcuma longa) titrated at 80% by weight of curcumin, and
less than 1000 mg, preferably less than 500 mg, more preferably less than 300 mg of quercetin.

According to a more preferred embodiment, the first part comprises:
about 5 g of glutamine,
about 18 IU (12 mg) of vitamin E, preferably natural vitamin E,
about 200 mg of dry extract of aerial parts of milk thistle (Silybum marianum) titrated at 80% by weight of silybin,
about 1.666 g of oat beta-glucans,
about 1.666 g of pectins, and
about 1.666 g of cellulose.

Optionally, the first part can also comprise:
about 1 g of turmeric rhizome extract (Curcuma longa) titrated at 80% by weight in curcumin, and
about 100 mg of quercetin.

Advantageously, the first part is presented in the form of a sachet.

According to one embodiment, the second part comprises:
a mixture of three probiotics: Lactobacillus rhamnosus GG, Bifidobacterium breve BR03 (DSM 16604), Lactobacillus plantarum LP01 (LMG P-21021), and
acacia fibers.

According to a preferred embodiment, the second part comprises:
less than 100 g, preferably less than 75 g, more preferably less than 55 g of a mixture of three probiotics: Lactobacillus rhamnosus GG, Bifidobacterium breve BR03 (DSM 16604), Lactobacillus plantarum LP01 (LMG P-21021), and
less than 500 mg, preferably less than 400 mg, more preferably less than 300 mg of acacia fibers.

According to a more preferred embodiment, the second part comprises:
about 50 mg (i.e., about 5 billion CFU) of a mixture of three probiotics: Lactobacillus rhamnosus GG, Bifidobacterium breve BR03 (DSM 16604), Lactobacillus plantarum LP01 (LMG P-21021), and
about 200 mg of acacia fiber.

Advantageously, the second part is presented in the form of a capsule, preferably a capsule consisting of hydroxypropylmethylcellulose, more preferably a capsule consisting of 75 mg of hydroxypropylmethylcellulose.

According to one embodiment, the third part comprises fish oil, preferably titrated to at least 60% by weight of omega-3, for example said omega-3 can comprise docosahexaenoic acid (DHA) and eicosapentaenoic acid (EPA).

Advantageously, the third part also comprises turmeric, or an extract of turmeric rhizome titrated from 80% to 95% by weight in curcumin, and quercetin.

Thus, the third part can for example comprise:
fish oil, preferably titrated to at least 60% by weight of omega-3, for example said omega-3 can comprise docosahexaenoic acid (DHA) and eicosapentaenoic acid (EPA);
turmeric or an extract of turmeric rhizome titrated from 80% to 95% by weight in curcumin; and
quercetin.

According to a preferred embodiment, the third part comprises less than 2000 mg, preferably less than 1500 mg, more preferably less than 1250 mg of fish oil, preferably titrated to at least 60% by weight of omega-3, for example said omega-3 can comprise docosahexaenoic acid (DHA) and eicosapentaenoic acid (EPA).

Advantageously, the third part also comprises:
less than 1.75 g, preferably less than 1.5 g, more preferably less than 1.35 g of turmeric rhizome extract (Curcuma longa) titrated at 80% by weight of curcumin, and
less than 1000 mg, preferably less than 500 mg, more preferably less than 300 mg of quercetin.

Thus, the third part can comprise:
less than 2000 mg, preferably less than 1500 mg, more preferably less than 1250 mg of fish oil, preferably titrated to at least 60% by weight of omega-3, for example said omega-3 may comprise docosahexaenoic acid (DHA) and eicosapentaenoic acid (EPA);
less than 1.75 g, preferably less than 1.5 g, more preferably less than 1.35 g of turmeric rhizome extract (Curcuma longa) titrated at 80% by weight of curcumin; and
less than 1000 mg, preferably less than 500 mg, more preferably less than 300 mg of quercetin.

According to a more preferred embodiment, the third part comprises about 1000 mg of fish oil, preferably titrated to at least 60% by weight of omega-3, for example said omega-3 can comprise docosahexaenoic acid (DHA) and eicosapentaenoic acid (EPA).

Advantageously, the third part also comprises:
about 1 g of turmeric rhizome extract (Curcuma longa) titrated at 80% by weight in curcumin, and
about 100 mg of quercetin.

According to an even more preferred embodiment, the third part comprises:
about 1000 mg of fish oil, preferably titrated to at least 60% by weight of omega-3, for example said omega-3 can comprise docosahexaenoic acid (DHA) and eicosapentaenoic acid (EPA);
about 1 g of turmeric rhizome extract (Curcuma longa) titrated at 80% by weight in curcumin; and
about 100 mg of quercetin.

Advantageously, the fish oil of the composition according to the invention comprises 60% by weight of omega-3, in particular about 30% by weight of docosahexaenoic acid (DHA) relative to the weight of the fish oil, about 20% by weight of eicosapentaenoic acid (EPA) relative to the weight of fish oil and about 10% by weight of omega-3 other than DHA and EPA relative to the weight of fish oil.

Advantageously, the third part further comprises glycerol, preferably about 182 mg of glycerol. Glycerol is an excipient used as a humectant.

Optionally, the third part also comprises at least one additive aimed at preventing the deterioration of fish oil, for example selected from: an extract of rosemary and a mixture of tocopherols; this at least one additive aimed at preventing the deterioration of the fish oil is present in a small amount (less than 5%, preferably less than 3%, more preferably less than or equal to 1% by weight relative to the weight of the third part) and is not intended to induce an antioxidant effect in the subject in whom the third part of the composition according to the invention is administered.

Advantageously, the third part is presented in the form of at least one soft capsule, preferably at least one soft capsule consisting of fish gelatin, more preferably at least one soft capsule whose structure consists of 182 mg of fish gelatin. The third part can for example also be presented in the form of 2 soft capsules.

According to an advantageous feature, when the first part of the composition comprises turmeric or an extract of turmeric rhizome titrated from 80% to 95% by weight of curcumin and quercetin, the third part of the composition does not comprise any; and conversely, when the third part of the composition comprises turmeric, or a turmeric rhizome extract titrated from 80% to 95% by weight of curcumin, and quercetin, the first part of the composition does not comprise any.

The invention relates in particular to said first, second and third parts of the composition according to the invention as described above, for their use in the treatment of non-alcoholic fatty liver disease (NAFLD). Advantageously, these three parts of the composition according to the invention are administered daily at the rate of a first, a second and two third parts per day.

Excipient

In one embodiment, the nutraceutical or pharmaceutical composition of the invention further comprises, in one or more of its parts where appropriate, at least one pharmaceutically or nutraceutically acceptable excipient.

The pharmaceutical or nutraceutical composition can typically comprise carriers or vehicles. The term “carriers” or “vehicles” refers to materials suitable for administration and includes any such material known in the art, such as, for example, any liquid, gel, solvent, liquid diluent, solubilizing agent or the like, non-toxic and which does not interact with any component of the composition in a detrimental way. Examples of pharmaceutically or nutraceutically acceptable carriers include, for example, water, saline solutions, alcohol, silicone, waxes, petrolatum, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, esters of fatty acids, hydroxymethylcellulose, hydroxypropylmethylcellulose polyvinylpyrrolidone, etc.

Thus, the nutraceutical or pharmaceutical composition may further comprise a texturing agent, preferably a gelling agent, even more preferably a modified starch.

The nutraceutical or pharmaceutical composition may further comprise an anti-sticking agent in order to improve the rheological properties of the pharmaceutical or nutraceutical composition.

In one embodiment, the anti-sticking agent is magnesium stearate.

In one embodiment, the nutraceutical or pharmaceutical composition further comprises minerals and micronutrients such as nutrient minerals and vitamins in accordance with recommendations from government bodies such as the USRDA. For example, the composition may contain per daily dose one or more of the following micronutrients: zinc, chromium, calcium, magnesium, phosphorus, iron, copper, iodine, selenium, beta-carotene, vitamin C, vitamin B1, vitamin B6, vitamin B2, niacin, vitamin B12, folic acid, biotin, vitamin D or vitamin E.

In one embodiment, the nutraceutical or pharmaceutical composition comprises:
a. one or more sources of dietary fibers selected from sources of vegetable fibers, preferably selected from sources of celluloses, hemicellulose, pectins, starch, beta-glucans, and a mixture of these compounds,
b. glutamine,
c. one or more probiotics,
d. one or more sources of omega-3, and
e. one or more antioxidants,
for use in the prevention and/or treatment of dysbiosis of the intestinal microbiota.

In one embodiment, the nutraceutical or pharmaceutical composition comprises:
a. one or more sources of dietary fibers,
b. glutamine,
c. one or more probiotics selected from lactic acid bacteria, preferably selected from the family of Lactobacillaceae, Bifidobacteriaceae, and a mixture thereof, more preferably selected from Lactobacillus rhamnosus, Lactobacillus plantarum, Bifidobacterium breve, and a mixture thereof,
d. one or more sources of omega-3, and
e. one or more antioxidants,
for use in the prevention and/or treatment of dysbiosis of the intestinal microbiota.

In one embodiment, the nutraceutical or pharmaceutical composition comprises:
a. one or more sources of dietary fibers,
b. glutamine,
c. one or more probiotics,
d. one or more sources of omega-3 selected from docosahexaenoic acid, eicosatetraenoic acid, hexadecatrienoic acid, α-linolenic acid, stearidonic acid, eicosatrienoic acid, eicosapentaenoic acid, heneicosapentaenoic acid, docosapentaenoic acid, tetracosapentaenoic acid, tetracosahexaenoic acid, and mixtures of these compounds, and preferably selected from docosahexaenoic acid, eicosatetraenoic acid, α-linolenic acid, and mixtures of these compounds, and
e. one or more antioxidants,
for use in the prevention and/or treatment of dysbiosis of the intestinal microbiota.

In one embodiment, the nutraceutical or pharmaceutical composition comprises:
a. one or more sources of dietary fibers,
b. glutamine,
c. one or more probiotics,
d. one or more sources of omega-3, and
e. one or more antioxidants selected from curcuminoids, quercetin, rutin, catechins, boswellic acid, resveratrol, berberine, silybin and a mixture of these compounds, and preferably selected from curcumin, quercetin, resveratrol, berberine, silybin, catechins, boswellic acid and a mixture of these compounds,
for use in the prevention and/or treatment of dysbiosis of the intestinal microbiota.

In one embodiment, the nutraceutical or pharmaceutical composition comprises:
a. one or more sources of dietary fibers selected from sources of vegetable fibers, preferably selected from sources of celluloses, hemicellulose, pectins, starch, beta-glucans, and a mixture of these compounds,
b. glutamine,
c. one or more probiotics selected from lactic acid bacteria, preferably selected from the family of Lactobacillaceae, Bifidobacteriaceae, and a mixture thereof, more preferably selected from Lactobacillus rhamnosus, Lactobacillus plantarum, Bifidobacterium breve, and a mixture thereof,
d. one or more sources of omega-3, and
e. one or more antioxidants,
for use in the prevention and/or treatment of dysbiosis of the intestinal microbiota.

In one embodiment, the nutraceutical or pharmaceutical composition comprises:
a. one or more sources of dietary fibers,
b. glutamine,
c. one or more probiotics,
d. one or more sources of omega-3 selected from docosahexaenoic acid, eicosatetraenoic acid, hexadecatrienoic acid, α-linolenic acid, stearidonic acid, eicosatrienoic acid, eicosapentaenoic acid, heneicosapentaenoic acid, docosapentaenoic acid, tetracosapentaenoic acid, tetracosahexaenoic acid, and mixtures of these compounds, and preferably selected from docosahexaenoic acid, eicosatetraenoic acid, α-linolenic acid, and mixtures of these compounds, and
e. one or more antioxidants selected from curcuminoids, quercetin, rutin, catechins, boswellic acid, resveratrol, berberine, silybin and a mixture of these compounds, and preferably selected from curcumin, quercetin, resveratrol, berberine, silybin, catechins, boswellic acid and a mixture of these compounds,
for use in the prevention and/or treatment of dysbiosis of the intestinal microbiota.

In one embodiment, the nutraceutical or pharmaceutical composition comprises:
a. one or more sources of dietary fibers selected from sources of vegetable fibers, preferably selected from sources of celluloses, hemicellulose, pectins, starch, beta-glucans, and a mixture of these compounds,
b. glutamine,
c. one or more probiotics selected from lactic acid bacteria, preferably selected from the family of Lactobacillaceae, Bifidobacteriaceae, and a mixture thereof, more preferably selected from Lactobacillus rhamnosus, Lactobacillus plantarum, Bifidobacterium breve, and a mixture thereof,
d. one or more sources of omega-3 selected from docosahexaenoic acid, eicosatetraenoic acid, hexadecatrienoic acid, α-linolenic acid, stearidonic acid, eicosatrienoic acid, eicosapentaenoic acid, heneicosapentaenoic acid, docosapentaenoic acid, tetracosapentaenoic acid, tetracosahexaenoic acid, and mixtures of these compounds, and preferably selected from docosahexaenoic acid, eicosatetraenoic acid, α-linolenic acid, and mixtures of these compounds, and
e. one or more antioxidants selected from curcuminoids, quercetin, rutin, catechins, boswellic acid, resveratrol, berberine, silybin and a mixture of these compounds, and preferably selected from curcumin, quercetin, resveratrol, berberine, silybin, catechins, boswellic acid and a mixture of these compounds,
for use in the prevention and/or treatment of dysbiosis of the intestinal microbiota.

Administration Route

In one embodiment, the composition according to the invention is orally administered.

In one embodiment, the composition according to the invention is presented in one or more of the following forms: capsules (hard capsules), soft capsules, tablets, drinkable solutions, powders to be dissolved and/or dispersed, concentrated solutions to be diluted, sachets (sealed bags).

In one embodiment, the composition according to the invention is presented in the form of capsules.

In one embodiment, the composition according to the invention is presented in the form of soft capsules.

In one embodiment, the composition according to the invention is presented in the form of a sachet.

In one embodiment, the first part of the composition according to the invention as defined hereinabove is presented in the form of a sachet.

In one embodiment, the second part of the composition according to the invention as defined hereinabove is presented in the form of a soft capsule. In another embodiment, the second part of the composition according to the invention as defined hereinabove is in the form of a hard capsule.

In one embodiment, the third part of the composition according to the invention as defined hereinabove is presented in the form of a hard capsule. In another embodiment, the third part of the composition according to the invention as defined hereinabove is presented in the form of a soft capsule.

Use of the Composition

The features of the composition according to the invention as described above apply for the uses of the composition, the composition for uses and for the methods of prevention and/or treatment comprising a step of administration to a patient in need of the composition according to the invention.

The composition according to the invention is used in the prevention and/or treatment of dysbiosis of the intestinal microbiota. An object of the present invention is therefore the composition according to the invention, for use in the prevention and/or treatment of dysbiosis of the intestinal microbiota. As mentioned above, dysbiosis includes a change in the intestinal flora (intestinal microbiota), increased intestinal permeability, oxidative stress, and low-grade inflammation. The composition according to the invention has the advantage of acting on these four axes of dysbiosis. Thus, the present invention also relates to the composition according to the invention for use for improving the intestinal microbiota, in particular by increasing intestinal bacterial diversity, for reducing intestinal permeability, for reducing oxidative stress and for reducing low-grade inflammation.

The composition according to the invention is thus also used in the preservation and/or restoration of the symbiosis of the intestinal microbiota. An object of the present invention is therefore the composition according to the invention, for use in the preservation and/or restoration of the symbiosis of the intestinal microbiota. Indeed, the composition according to the invention allows the preservation and/or restoration of the symbiosis through the prevention and/or treatment of dysbiosis of the intestinal microbiota.

In one embodiment, the dysbiosis of the intestinal microbiota is associated with Irritable Bowel Syndrome (IBS).

In one embodiment, the dysbiosis of the intestinal microbiota is associated with type I diabetes.

In one embodiment, the dysbiosis of the intestinal microbiota is associated with type II diabetes.

In one embodiment, the dysbiosis of the intestinal microbiota is associated with obesity.

In one embodiment, the dysbiosis of the intestinal microbiota is associated with non-alcoholic fatty liver disease (NAFLD).

In one embodiment, the dysbiosis of the intestinal microbiota is associated with Crohn's disease.

In one embodiment, the dysbiosis of the intestinal microbiota is associated with Irritable Bowel Syndrome, type I diabetes, type II diabetes, obesity, non-alcoholic fatty liver disease, and Crohn's disease.

In one embodiment, the dysbiosis of the intestinal microbiota is associated with Irritable Bowel Syndrome, type I diabetes, type II diabetes, obesity, non-alcoholic fatty liver disease.

In one embodiment, the dysbiosis of the intestinal microbiota is associated with Irritable Bowel Syndrome (IBS)

In one embodiment, the dysbiosis of the intestinal microbiota is associated with allergies.

In one embodiment, dysbiosis of the intestinal microbiota is associated with food-type allergies.

In one embodiment, the composition according to the invention is used in the prevention and/or treatment of Irritable Bowel Syndrome (IBS). An object of the present invention is thus the composition according to the invention in the prevention and/or treatment of Irritable Bowel Syndrome (IBS).

In one embodiment, the composition according to the invention is used in the prevention and/or treatment of type I diabetes. An object of the present invention is thus the composition according to the invention in the prevention and/or treatment of type I diabetes.

In one embodiment, the composition according to the invention is used in the prevention and/or treatment of type II diabetes. An object of the present invention is thus the composition according to the invention in the prevention and/or treatment of type II diabetes.

In one embodiment, the composition according to the invention is used in the prevention and/or treatment of obesity. An object of the present invention is thus the composition according to the invention in the prevention and/or treatment of obesity.

In one embodiment, the composition according to the invention is used in the prevention and/or treatment of non-alcoholic fatty liver disease (NAFLD). An object of the present invention is thus the composition according to the invention in the prevention and/or treatment of non-alcoholic fatty liver disease (NAFLD). Indeed, currently, the only effective treatment for NAFLD is to reduce the weight of the patient by at least 10%. However, this loss of weight objective is difficult to maintain over the long term and other actions should be considered. Dysbiosis as well as insulin resistance are implicated in the pathophysiology of NAFLD. Thus, the present invention also relates to the composition according to the invention for use in the prevention and/or treatment of NAFLD.

In one embodiment, the composition according to the invention is used in the prevention and/or treatment of Crohn's disease. An object of the present invention is thus the composition according to the invention in the prevention and/or treatment of Crohn's disease.

The invention also relates to a method of preventing and/or treating dysbiosis comprising a step of administering to a patient in need thereof the composition according to the invention.

In one embodiment, the invention also relates to a method of preventing and/or treating Irritable Bowel Syndrome (IBS) comprising a step of administering to a patient in need thereof the composition according to the invention.

In one embodiment, the invention also relates to a method of preventing and/or treating type I diabetes comprising a step of administering to a patient in need thereof the composition according to the invention.

In one embodiment, the invention also relates to a method of preventing and/or treating type II diabetes comprising a step of administering to a patient in need thereof the composition according to the invention.

In one embodiment, the invention also relates to a method of preventing and/or treating obesity comprising a step of administering to a patient in need thereof the composition according to the invention.

In one embodiment, the invention also relates to a method of preventing and/or treating non-alcoholic fatty liver disease comprising a step of administering to a patient in need thereof the composition according to the invention.

In one embodiment, the invention also relates to a method of preventing and/or treating Crohn's disease comprising a step of administering to a patient in need thereof the composition according to the invention.

In one embodiment, the composition according to the invention is administered once daily.

In one embodiment, the composition according to the invention is administered twice daily.

In one embodiment, the composition according to the invention is administered daily over a period of between 1 and 3 months.

In one embodiment, the composition according to the invention is administered daily over a period of more than 3 months, in particular over a period of at least 12 months.

Kit

The present invention also relates to a kit in three parts:
a first part comprising one or more sources of dietary fibers, preferably as defined above, glutamine, preferably as defined above,
a second part comprising one or more probiotics, preferably as defined above,
a third part comprising one or more sources of omega-3, preferably as defined above,
one or more antioxidants, preferably as defined above, the antioxidant(s) being comprised in the first part of the kit and/or in the second part of the kit.

The different parts of the kit can have embodiments corresponding to the different embodiments defined for the first, second and third parts of the composition according to the invention, as defined hereinabove.

### EXAMPLES

The present invention will be better understood in view of the following examples which illustrate without limitation the invention.

Examples of compositions according to the invention were prepared according to Tables 1 to 4 below.

### Example 1: Composition for Use in the Prevention and/or Treatment of Irritable Bowel Syndrome (IBS)

TABLE 1

| | | Purity/source of | Amount |
|---|---|---|---|
| Part | Ingredient | the ingredient | (mg*) |
| 1 (sachet) | Beta-glucan | 100% | 1666 |
| | Cellulose | 100% | 1666 |
| | Pectin | 100% | 1666 |
| | Glutamine | 100% | 5000 |
| | Resveratrol | 100% | 200 |
| | Boswellic acid | From Boswellia titrated at | 160 |
| | | 80% by weight of boswellic | |
| | | acid | |
| 2 (capsule) | Lactobacillus | Each strain is dosed at 100 | 50 |
| | rhamnosus GG + | billion CFU per gram. The | |
| | Bifidobacterium | proportion of each strain is | |
| | breve BR03 + | equal. The product contains | |
| | Lactobacillus | 5 billion CFU | |
| | LP01 plantarum | | |
| 3 (soft | Omega-3 | From fish oil titrated to 60% | 600 |
| capsule) | | by weight of omega-3 | |
| | | (33% EPA and 22% DHA) | |
| | Curcumin | From turmeric titrated at | 950 |
| | | 95% by weight in curcumin | |
| | Quercetin | From Sophora Japonica | 100 |
| | | titrated to 98% by weight of | |
| | | quercetin | |

*relative to the pure ingredient
EPA: Eicosapentaenoic acid
DHA: Docosahexaenoic acid

The three-part composition according to Table 1 above can be used in the treatment and/or prevention of Irritable Bowel Syndrome (IBS). The composition is administered orally in a single dose, daily, for a period of between 1 and 3 months, to patients with IBS.

The three-part composition according to Table 1 is expected to treat IBS.

### Example 2: Composition for Use in the Prevention and/or Treatment of Obesity

TABLE 2

| | | Purity/source of | Amount |
|---|---|---|---|
| Part | Ingredient | the ingredient | (mg*) |
| 1 (sachet) | Beta-glucan | 100% | 1666 |
| | Cellulose | 100% | 1666 |
| | Pectin | 100% | 1666 |
| | Glutamine | 100% | 5000 |
| | Catechin | From green tea titrated to | 60 |
| | | 30% by weight of catechins | |
| 2 (capsule) | Lactobacillus | Each strain is dosed at 100 | 50 |
| | rhamnosus GG + | billion CFU per gram. The | |
| | Bifidobacterium | proportion of each strain is | |
| | breve BR03 + | equal. The product contains | |
| | Lactobacillus | 5 billion CFU | |
| | LP01 plantarum | | |
| 3 (soft | Omega-3 | From fish oil titrated to 60% | 600 |
| capsule) | | by weight of omega-3 | |
| | | (33% EPA and 22% DHA) | |
| | Curcumin | From turmeric titrated at | 950 |
| | | 95% by weight in curcumin | |
| | Quercetin | From Sophora Japonica | 100 |
| | | titrated to 98% by weight of | |
| | | quercetin | |

*relative to the pure ingredient
EPA: Eicosapentaenoic acid
DHA: Docosahexaenoic acid

The three-part composition according to Table 2 above can be used in the treatment and/or prevention of obesity. The composition is administered orally in a single dose, daily, for a period of between 1 and 3 months, to obese patients.

The three-part composition according to Table 2 is expected to treat obesity.

### Example 3: Composition for Use in the Prevention and/or Treatment of Diabetes (Type I or II)

TABLE 3

| | | Purity/source of | Amount |
|---|---|---|---|
| Part | Ingredient | the ingredient | (mg*) |
| 1 (sachet) | Beta-glucan | 100% | 1666 |
| | Cellulose | 100% | 1666 |
| | Pectin | 100% | 1666 |
| | Glutamine | 100% | 5000 |
| | Berberine | 100% | 200 |
| 2 (capsule) | Lactobacillus | Each strain is dosed at 100 | 50 |
| | rhamnosus GG + | billion CFU per gram. The | |
| | Bifidobacterium | proportion of each strain is | |
| | breve BR03 + | equal. The product contains | |
| | Lactobacillus | 5 billion CFU | |
| | LP01 plantarum | | |
| 3 (soft | Omega-3 | From fish oil titrated to 60% | 600 |
| capsule) | | by weight of omega-3 | |
| | | (33% EPA and 22% DHA) | |
| | Curcumin | From turmeric titrated at | 950 |
| | | 95% by weight in curcumin | |
| | Quercetin | From Sophora Japonica | 100 |
| | | titrated to 98% by weight of | |
| | | quercetin | |

*relative to the pure ingredient
EPA: Eicosapentaenoic acid
DHA: Docosahexaenoic acid

The three-part composition according to Table 3 above can be used in the treatment and/or prevention of type I or II diabetes. The composition is administered orally in a single dose, daily, for a period of between 1 and 3 months, to patients with diabetes.

The three-part composition according to Table 3 is expected to treat type I or II diabetes.

### Example 4: Composition for Use in the Prevention and/or Treatment of Non-Alcoholic Fatty Liver Disease (NAFLD)

TABLE 4

| | | Purity/source of | Amount |
|---|---|---|---|
| Part | Ingredient | the ingredient | (mg*) |
| 1 (sachet) | Beta-glucan | 100% | 1666 |
| | Cellulose | 100% | 1666 |
| | Pectin | 100% | 1666 |
| | Glutamine | 100% | 5000 |
| | Silybin | From milk thistle titrated to | 200 |
| | | 80% by weight of silybin | |
| 2 (capsule) | Lactobacillus | Each strain is dosed at 100 | 50 |
| | rhamnosus GG + | billion CFU per gram. The | |
| | Bifidobacterium | proportion of each strain is | |
| | breve BR03 + | equal. The product contains | |
| | Lactobacillus | 5 billion CFU | |
| | LP01 plantarum | | |
| 3 (soft | Omega-3 | From fish oil titrated to 60% | 600 |
| capsule) | | by weight of omega-3 | |
| | | (33% EPA and 22% DHA) | |
| | Curcumin | From turmeric titrated at | 950 |
| | | 95% by weight in curcumin | |
| | Quercetin | From Sophora Japonica | 100 |
| | | titrated to 98% by weight of | |
| | | quercetin | |

*relative to the pure ingredient
EPA: Eicosapentaenoic acid
DHA: Docosahexaenoic acid

The three-part composition according to Table 4 above can be used in the treatment and/or prevention of non-alcoholic fatty liver disease (NAFLD). The composition is administered orally in a single dose, daily, for a period of between 1 and 3 months, to patients with NAFLD.

The three-part composition according to Table 4 is expected to treat NAFLD.

Thus, through these four examples, it is expected that the compositions according to the examples above restore dysbiosis by the combined action of the constituents on various manifestations of dysbiosis (decrease in bacterial diversity, modification of intestinal permeability, increased oxidative stress, inflammation). The action on these different levers makes it possible to treat dysbiosis and restore the symbiosis of the microbiota and the body.

### Example 5: Observational Study of the Efficacy of a Composition According to the Invention in Improving Moderate to Severe Non-Alcoholic Fatty Liver Disease (NAFLD)

The severity of NAFLD is related to the extent of the liver fibrosis; it can progress to cirrhosis (stage F4 fibrosis according to the METAVIR score), while cirrhosis can itself evolve to hepatocarcinoma (primary liver cancer). Currently, the only effective treatment for NAFLD is to reduce the weight of the patient by at least 10%. However, this weight loss objective is difficult to maintain over the long term and other actions should be considered. A double-blind observational study is thus carried out in order to compare the effectiveness of a composition according to the invention, hereinafter called “Combo”, with a placebo, in improving moderate to severe NAFLD.

Materials and Methods

Materials

Composition of the capsule of Combo and the placebo capsule:

| Capsule | Combo | Placebo |
|---|---|---|
| Probiotics: | 50 mg or | — |
| Lactobacillus rhamnosus GG | 5 billion | |
| Bifidobacterium breve BR03 (DSM 16604) | CFU | |
| Lactobacillus plantarum LP01 (LMG P-21021) | | |
| Shell: HPMC | 75 mg | 75 mg |
| Acacia fibers | 200 mg | — |
| Maltodextrine | — | 250 mg |

Composition of the soft capsule of Combo and the placebo soft capsule:

| Soft capsule | Combo | Placebo |
|---|---|---|
| Fish oil titrated at 60% by weight in omega-3 | 500 mg | — |
| Shell: fish gelatin | 182 mg | 162 mg |
| Moistening agent: glycerol | | 53 mg |
| Additives to prevent spoilage of fish oil: mixture of | | — |
| tocopherols (E306), rosemary extract (E392) | | |
| Soy lecithin | — | 540 mg |

The additives to prevent spoilage of fish oil (mixture of tocopherols (E306) and rosemary extract (E392)) are present only as excipients and are only intended to prevent oxidation of the fish oil.

Composition of the sachet of Combo and the placebo sachet:

| Sachet | Combo | | Placebo |
|---|---|---|---|
| Glutamine | 5 | g | — |
| Turmeric rhizome extract (Curcuma longa) | 1 | g | — |
| titrated at 80% by weight in curcumin | | | |
| Natural Vitamin E | 12 mg (18 UI) | | — |
| Quercetin | 100 | mg | — |
| Dry extract of aerial parts of milk thistle | 200 | mg | — |
| (Silybum marianum) titrated at 80% by | | | |
| weight in silybin | | | |
| Oat beta-glucans from oat bran (Avena saliva) | 1.666 | g | — |
| Pectin | 1.666 | g | — |
| Cellulose | 1.666 | g | — |

| | | | |
|---|---|---|---|
| Maltodextrine | — | 9 | g |
| Talc | — | 2 | g |
| Orange Yellow S (E110) | — | 10 | mg |

The placebo is identical in shape, color, presentation and taste compared to the Combo. The Combo and the placebo are provided to patients in one-month boxes containing 30 white sachets and 60 soft capsules and 30 capsules in blister packs.

60 patients are included in the study. These patients have moderate to severe diagnosed NAFLD and moderate to severe fibrosis (stage F2 or F3 according to the METAVIR score) and are 18 years or older. They are offered inclusion in the study when their liver biopsy results are announced. The criteria for not including people who are willing to participate to research are as follows: cirrhosis (stage F4 according to the METAVIR score); hepato-cellular carcinoma; ongoing steatogenic treatment (corticosteroids, methotrexate, amiodarone, tamoxifen) or hepatotoxic treatment (amitriptyline, imipramine, clozapine, diclofenac); viral liver damage; autoimmune liver damage; anticoagulant therapy; antibiotic treatment in the two months preceding inclusion; allergy to soy, aspirin, fish, maltodextrin or E110; poorly balanced diabetes (HbA1c>8%); pregnant woman; excessive alcohol intake (>100 g/week).

Methods

Randomization is carried out electronically via a dedicated website. Thus, independently of the doctors and the patients, each patient is included either in the group of patients receiving the Combo or in the group of patients receiving the placebo.

30 patients receive the Combo and 30 patients receive the placebo, for 48 weeks for each patient. Each patient receives the quantity needed for three months of Combo or placebo on a quarterly basis from the pharmacy of one of the two centers participating in the research.

Administration method: each patient administers to themselves each day, once a day, in the morning: 2 soft capsules, 1 capsule and 1 sachet diluted in a 100 mL glass of water or fruit juice or in a food product, whether it is the Combo or the placebo. This administration is carried out every day for 48 weeks for each patient.

In addition, physical activity advice and hygiene and dietetic advice are provided to each included patient.

Patient follow-up methods:
Medical visits: 6 medical visits for clinical examination are carried out: the day of inclusion (W0), 4 weeks after inclusion (W4), 12 weeks after inclusion (W12), 24 weeks after inclusion (W24), 36 weeks after inclusion (W36) and at the end of the study (at the end of the 48th week: W48). The clinical parameters checked during each of the 6 visits are as follows: weight, blood pressure, abdominal perimeter, BMI calculation. In addition, an elastometric measurement by FibroScan® is carried out at W0, W24 and W48.
Biological controls: blood samples and stool samples are taken to assess the safety and efficacy of the Combo: on inclusion in the study (W0), 24 weeks after inclusion (W24) and at the end of the study (W48).
   Blood samples: the biological parameters checked are as follows: ASAT, ALAT, alkaline phosphatase, gamma-GT, total bilirubin, triglycerides, HDL-cholesterol, LDL-cholesterol, apolipoprotein A1, ferritinemia, glycemia, glycated hemoglobin (HbA1c), insulinemia, calculation of the HOMA index (Homeostasis Model Assessment of insulin resistance), platelets, C-reactive protein (CRP), ionogram (natremia, kalaemia), serum creatinine, albuminemia, alpha2macroglobulinemia, haptoglobulinemia, calculation of the Fibrosis 4 (FIB4) score, calculation of the NAFLD score, oxidative stress measured by the analysis method of the company “Laboratoires Réunis” (which involves the measurement of antioxidant capacity), lipopolysaccharides (LPS), C-reactive protein (CRP), IL6 (interleukin-6), TNF (tumor necrosis factor);
   Stool samples: the parameter analyzed is as follows: analysis of the fecal microbiota by sequencing of the 16S RNA of bacteria (Luxia “Phase IV” method);
Anatomic pathology: a liver biopsy puncture is performed at the end of the study (W48) with calculation of the SAF score, in addition to the liver biopsy puncture performed before the inclusion of patients in the study;
Dietetic assessment and IPAQ questionnaire: a dietetic assessment and a response to an IPAQ questionnaire by each patient are scheduled three times during the study: the day of inclusion (W0), 24 weeks after inclusion (W24) and at the end of the study (W48).

In some cases, treatment should be interrupted: serious side effect, worsening of liver disease justifying stopping the treatment, antibiotic therapy for more than one month, decision of the patient.

The reasons for patients leaving the study are: withdrawal of the consent of the patient (possible at any time during the study, without justification required), loss of follow-up of patients, death, antibiotic therapy for more than one month.

The objectives of administering the Combo are the following:
main objective: reduction of NAFLD at W48 with reduction or stabilization of histological signs, reduction in elastometric scores and in NAFLD and FIB4 biological scores; and
secondary objectives: improvement of the symbiosis and improvement of the quality of life (physical and nutritional state) at W48.

The outcomes for the study are as follows:
the criteria for assessment of the main outcome are:
   histological improvement in NAFLD after 48 weeks of taking the Combo: a liver biopsy puncture to assess the histological evolution of NAFLD is performed at W48. A histological improvement in NAFLD is expected with an interruption in the progression of NAFLD, at least a stabilization or even a decrease in fibrosis and a decrease in fibrinogenesis. The SAF score is calculated (Steatosis Activity Fibrosis); it is estimated at 4.5 on the day of inclusion (W0) and it is expected that it will be 3.5 at the end of the study (W48), i.e., an improvement in the score of 30% and a decrease the median score of 1.0;
   a reduction in elastometry parameters: an examination by FibroScan® is performed at W48: evaluation of fibrosis by measuring elasticity and evaluation of steatosis by measuring the CAP (Controlled Attenuation Parameter). A decrease in elasticity of 2.5 kPa is expected (estimated elasticity before treatment: 9.5 kPa; expected elasticity after treatment: 7.0 kPa, i.e., a 26% improvement in elasticity). A decrease in CAP is expected from 330 dB/m to 247 dB/m, i.e., an improvement of 25%; and
   a decrease in FIB4 (Fibrosis-4 score) and NAFLD biological scores;
the secondary outcomes are:
   evaluation of the symbiosis at W48: a concomitant improvement of the microbiota (promotion of its diversity and its anti-inflammatory potential by increasing the relative abundance of butyrate-producing bacteria) and/or intestinal permeability and/or reduction of low-grade inflammation and/or reduction of oxidative stress. The parameters evaluated are: analysis of the microbiota in the stool (sequencing of 16S RNA from bacteria), measurement of intestinal permeability (determination of LPS lipopolysaccharides from a blood sample), measurement of low-grade inflammation (determination of inflammation mediators: CRP, IL6 and TNF from a blood sample), measurement of oxidative stress (antioxidant capacity from a blood sample). A concomitant improvement in microbiota parameters (relative abundance of bacteria producing butyrate) and/or intestinal permeability (decrease in LPS concentration) and/or low-grade inflammation (decrease in CRP, IL6 and/or TNF concentrations) and/or oxidative stress (increased antioxidant capacity) is expected; and
   lifestyle assessment at W48: physical activity (IPAQ questionnaire), nutritional status (summary over 3 days).

Statistical Analysis Plan: Statistical analysis is performed at the end of follow-up for all patients, after a blind review for data quality issues and a database freeze. The comparative analysis is carried out blind, that is to say that neither the statistician nor the coordinating investigator can identify the placebo and Combo groups. Once the analysis is finalized and presented to the steering committee, the identification of groups is provided. Statistical analysis is performed with a bilateral alpha risk of 5%.

Quantitative data is described by means and standard deviations and medians and interquartile ranges. They are compared using non-parametric tests (Wilcoxon Mann-Withney, Kruskal-Wallis). Paired tests are used when comparing repeated measurements of the same patients (evolution of biological data, elastometry, IPAQ).

Qualitative data are expressed in numbers and percentages. The proportions of missing data are calculated. They are compared using Chi-2 tests or Fisher tests when the groups are independent and McNemar tests when the groups are paired (repeated measures).

First, the data is analyzed by intention to treat in order to meet the primary and secondary endpoints.

All patients who received at least one administration of the study product are included in the analysis for the primary endpoint (ICH standards). The number of subjects to be included takes into account possible patients lost to follow-up. For the other analyzes and in the event of missing data, the values of the last known observation are used in the intention-to-treat analysis (LOCF method, last observation carried forward).

1-10. (canceled)

11. Nutraceutical or pharmaceutical composition comprising:
a. one or more sources of dietary fibers,
b. glutamine,
c. one or more probiotics,
d. one or more sources of omega-3, and
e. one or more antioxidants,
for use in the prevention and/or treatment of dysbiosis of the intestinal microbiota.

12. The composition according to claim 11, wherein the source(s) of dietary fibers are selected from sources of vegetable fibers, preferably are selected from sources of celluloses, hemicellulose, pectins, starch, beta-glucans, and a mixture of these compounds.

13. The composition according to claim 11, wherein the probiotic(s) are selected from lactic acid bacteria, preferably are selected from the family of Lactobacillaceae, Bifidobacteriaceae, and a mixture thereof, and more preferably are selected from Lactobacillus rhamnosus, Lactobacillus plantarum, Bifidobacterium breve, and a mixture thereof.

14. The composition according to claim 11, wherein the source(s) of omega-3 are selected from docosahexaenoic acid, eicosatetraenoic acid, hexadecatrienoic acid, α-linolenic acid, stearidonic acid, eicosatrienoic acid, eicosapentaenoic acid, heneicosapentaenoic acid, docosapentaenoic acid, tetracosapentaenoic acid, tetracosahexaenoic acid, and mixtures of these compounds, and preferably are selected from docosahexaenoic acid, eicosatetraenoic acid, α-linolenic acid, and mixtures of these compounds.

15. The composition according to claim 11, wherein the antioxidant(s) are selected from curcuminoids, quercetin, rutin, catechins, boswellic acid, resveratrol, berberine, silybin and a mixture of these compounds, and preferably are selected from curcumin, quercetin, resveratrol, berberine, silybin, catechins, boswellic acid and a mixture of these compounds.

16. The composition according to claim 11, said composition being in three parts, a first part comprising the source(s) of dietary fibers and glutamine, a second part comprising the probiotic(s), a third comprising the source(s) of omega-3, the antioxidant(s) being included in the first part of the composition and/or in the third part of the composition, preferably included in the first part and in the third part of the composition.

17. The composition according to claim 16, wherein the first part of the composition has a total content of sources of fibers ranging from 30 to 70% by weight, preferably ranging from 40 to 60% by weight, relative to the total weight of the first part of the composition.

18. The composition according to claim 16, wherein the first part of the composition has a total content of glutamine ranging from 30 to 70% by weight, preferably ranging from 40 to 60% by weight, relative to the total weight of the first part of the composition.

19. The composition according to claim 16, wherein the third part of the composition has a total content of sources of omega-3 ranging from 10 to 50% by weight, preferably ranging from 20 to 40% by weight, relative to the total weight of the third part of the composition.

20. The composition according to claim 11, wherein the dysbiosis of the intestinal microbiota is associated with Irritable Bowel Syndrome (IBS), type I diabetes, type II diabetes, obesity, non-alcoholic fatty liver disease (NAFLD) and Crohn's disease, preferably associated with Irritable Bowel Syndrome (IBS), type I diabetes, type II diabetes, obesity and non-alcoholic fatty liver disease (NAFLD), and more preferably associated with Irritable Bowel Syndrome (IBS).

21. A method of preventing and/or treating dysbiosis of the intestinal microbiota in a patient comprising administering the nutraceutical or pharmaceutical composition according to claim 11 to a patient in need thereof.

22. The method according to claim 21, wherein the source(s) of dietary fibers of the nutraceutical or pharmaceutical composition are selected from sources of vegetable fibers, preferably are selected from sources of celluloses, hemicellulose, pectins, starch, beta-glucans, and a mixture of these compounds.

23. The method according to claim 21, wherein the probiotic(s) of the nutraceutical or pharmaceutical composition are selected from lactic acid bacteria, preferably are selected from the family of Lactobacillaceae, Bifidobacteriaceae, and a mixture thereof, and more preferably are selected from Lactobacillus rhamnosus, Lactobacillus plantarum, Bifidobacterium breve, and a mixture thereof.

24. The method according to claim 21, wherein the source(s) of omega-3 of the nutraceutical or pharmaceutical composition are selected from docosahexaenoic acid, eicosatetraenoic acid, hexadecatrienoic acid, α-linolenic acid, stearidonic acid, eicosatrienoic acid, eicosapentaenoic acid, heneicosapentaenoic acid, docosapentaenoic acid, tetracosapentaenoic acid, tetracosahexaenoic acid, and mixtures of these compounds, and preferably are selected from docosahexaenoic acid, eicosatetraenoic acid, α-linolenic acid, and mixtures of these compounds.

25. The method according to claim 21, wherein the antioxidant(s) of the nutraceutical or pharmaceutical composition are selected from curcuminoids, quercetin, rutin, catechins, boswellic acid, resveratrol, berberine, silybin and a mixture of these compounds, and preferably are selected from curcumin, quercetin, resveratrol, berberine, silybin, catechins, boswellic acid and a mixture of these compounds.

26. The method according to claim 21, said nutraceutical or pharmaceutical composition being in three parts, a first part comprising the source(s) of dietary fibers and glutamine, a second part comprising the probiotic(s), a third comprising the source(s) of omega-3,
the antioxidant(s) being included in the first part of the nutraceutical or pharmaceutical composition and/or in the third part of the nutraceutical or pharmaceutical composition, preferably included in the first part and in the third part of the nutraceutical or pharmaceutical composition.

27. The method according to claim 26, wherein the first part of the nutraceutical or pharmaceutical composition has a total content of sources of fibers ranging from 30 to 70% by weight, preferably ranging from 40 to 60% by weight, relative to the total weight of the first part of the nutraceutical or pharmaceutical composition.

28. The method according to claim 26, wherein the first part of the nutraceutical or pharmaceutical composition has a total content of glutamine ranging from 30 to 70% by weight, preferably ranging from 40 to 60% by weight, relative to the total weight of the first part of the nutraceutical or pharmaceutical composition.

29. The method according to claim 26, wherein the third part of the nutraceutical or pharmaceutical composition has a total content of sources of omega-3 ranging from 10 to 50% by weight, preferably ranging from 20 to 40% by weight, relative to the total weight of the third part of the nutraceutical or pharmaceutical composition.

30. The method according to claim 21, wherein the dysbiosis of the intestinal microbiota is associated with Irritable Bowel Syndrome (IBS), type I diabetes, type II diabetes, obesity, non-alcoholic fatty liver disease (NAFLD) and Crohn's disease, preferably associated with Irritable Bowel Syndrome (IBS), type I diabetes, type II diabetes, obesity and non-alcoholic fatty liver disease (NAFLD), and more preferably associated with Irritable Bowel Syndrome (IBS).
